(19) United States
(12) Patent Application Publication
(10) Pub. No.: US 2004/0211156 A1
(43) Pub. Date: Oct. 28, 2004

(54) Method and device for manufacturing tubular packs filled with a material as well as tubular pack filled with a material

(75) Inventor: Frans Vermee, Remagen (DE)

Correspondence Address: GRIFFIN & SZIPL, PC, SUITE PH-1, 2300 NINTH STREET, SOUTH, ARLINGTON, VA, 22204, US

(73) Assignee: Frans Vermee GmbH, Remagen (DE)

(21) Appl. No.: 10/824,473

(22) Filed: Apr. 15, 2004

(30) Foreign Application Priority Data
Apr. 24, 2003 (DE) 10318511.9

(51) Int. Cl. A61B 17/28; A61B 17/42; A61B 17/44

(52) U.S. Cl. 53/430; 206/459.5

(57) ABSTRACT

For manufacturing tubular packs (16) filled with a material, a tube (14) filled with a material is first wound with an embracing means (24) in at least two displacement areas (22). The winding of the filled tube (14) with the embracing means (24) is effected over at least an entire circumference so that an embracing angle of more than 360° is formed, precisely one winding area (40) being produced in the respective displacement area (22). Since the holding force of the embracing means (24) clearly increases with the embracing angle, the strength of the winding in the displacement area (22) of the tube (14) can be increased by selecting a correspondingly large embracing angle whereby the tightness is improved. Further, the embracing means (24) is fixed in the displacement area (22) so that the embracing means (24) cannot unwind and the tubular pack (16) is tightly sealed. Thus, a particularly tight seal of the tubular pack (16) is produced by winding which also remains tight by fixing.

### BACKGROUND OF THE INVENTION

1. Field of the Invention

The invention relates to a method and a device for manufacturing tubular packs filled with a material, particularly a food or chemical products (silicones, adhesives, explosives) as well as such a tubular pack. Such tubular packs, for example, are used for packing spreadable sausage and the like. Further; such tubular packs are suitable for packing chemical products, particularly silicones and adhesives as well as for packing explosives.

Usually, tubular packs are filled with the respective material and closed by a metal sealing clamp at regular intervals. The tubular pack is an endless material so that the sealing clamps are arranged such that between two successive sealing clamps, a tubular pack filled with a material and a non-filled tubular section are alternately formed. To manufacture a separate tubular pack filled with a material, the tubular pack is separated by cutting in the non-filled section after it has been sealed by sealing clamps.

2. Description of Related Art

A device for manufacturing tubular packs filled with a food is described in German Patent DE 40 11 470. For sealing the individual tubular packs, sealing clamps consisting of a flat metal piece are employed. The metal piece which is rectangular in the non-deformed state is laid about the filled tubular pack and closed by a closing means. By means of the closing means, the sealing clamp is bent and pressed together from two sides so that a flat sealing clamp is created. The ends of the sealing clamp overlap to avoid that the material in the tubular pack leaks out. By bending the sealing clamp, two bows are created which almost form a semicircle. Because of the resilience of the metallic sealing clamp, this does not result in a complete semicircle since the sealing clamp ends can only be bent parallel to each other at maximum and slightly spring back thereafter. Thus, the sealing clamp forms a cross section that is slightly triangular.

Such sealing clamps have the disadvantage that tightness problems often occur, particularly because of the overlapping ends of the sealing clamps. This leads to that the preservability of such products is limited and the products have to be stored in cold storage shelves. Moreover, the function of the overlapping part of the sealing clamp substantially consists in preventing the sealing clamp from bending up completely. Not least because of the resiliency of the sealing clamp, the overlapping part of the sealing clamp does not exert any radial force upon the part pressed together so that this part does not make any contribution to the actual sealing of the tubular pack.

Further, the combination of a metallic sealing clamp with a plastic tubular pack makes the recycling more difficult-since the material to be utilized is not of the same kind. Another disadvantage consists in that only sealing clamps of a certain size can be processed by the device described. This results in that only tubular packs within a small range of diameters can be processed. The processable dimensional tolerances are extremely small.

Further, a processing of different packaging materials is difficult if they have different material thicknesses and, as a consequence, in the gathered state, a different amount of material occurs at that location where the sealing clamp is arranged. Particularly with tubular packs with a small material thickness, this very often results in tightness problems since non-sealed cross-sectional surfaces are created. To be able to process different tubular packs with this device, very troublesome modifications are required.

### SUMMARY OF THE INVENTION

It is the object of the present invention to provide a method and a device for manufacturing tubular packs filled with a material by means of which different tubular packs particularly having a small material thickness can be sealed tightly. Further, it is an object of the invention to provide a tubular pack the tightness of which is improved.

This object is solved, according to the invention, with a method according to claim 1, a device according to claim 9 as well as with a tubular pack according to claim 15.

In the method for manufacturing tubular packs filled with a material according to the invention, a tube filled with the material is initially wound with an embracing means in at least two displacement areas. The winding of the tube with the embracing means is effected over at least an entire circumference so that a winding angle of more than 360° is formed. Through the area within the displacement area in which the embracing means is arranged, a winding area is defined. Since the holding force of the embracing means strongly increases with the winding angle, the strength of the winding in the displacement or winding area of the tube can be increased by a correspondingly high number of embracings whereby the tightness is improved. The embracing means is further fixed in the displacement area so that the embracing means is not able to unwind so that the tubular pack is tightly sealed. The embracing means is particularly fixed in the winding area. Thus, a particularly tight sealing of the tubular pack is created by winding, which remains tight, too, by fixing.

Since the tubular pack filled with the material can be strongly constricted by the method according to the invention, the tightness of the tubular pack is clearly increased. Thereby, the stock keeping can be improved since it can possibly be done totally without cooling. Particularly With foodstuff, the increased tightness results in that the preservability is increased. Further, the method according to the invention can be employed in an extremely variable manner since tubular packs with extremely different diameters can be processed.

The embracing means may have a cord-like configuration so that the length of the embracing means can be individually adapted, particularly automatically, in dependence on the application. As tubular envelope, a great variety of materials with a great variety of material thicknesses can be particularly used since the material used can always be automatically gathered into the state of maximum or desired tightness. Irregular surfaces or material bulgings or the like hardly have any influence upon the tightness of the tubular pack. Particularly if the embracing means consists of a material similar to that of the tube used, the recycling of the tubular pack manufactured according to the method according to the invention can be clearly simplified since the material to be utilized is largely of the same kind. With a cord-shaped embracing means, the displacement area in which there is substantially no material can be kept relatively narrow in comparison with the use of metallic sealing clamps. Further, it is possible to seal two ends of two different tubular packs with a single embracing means. In contrast to the use of two metallic sealing clamps in the area of displacement, one operational step is saved whereby the manufacture of tubular packs can be effected more rapidly. Thereby, the method according to the invention is particularly suitable for the mass production.

It is particularly provided that substantially no material is left in the displacement area so that the tubular pack is sealed as tightly as possible. This can be achieved by additionally displacing the material in the displacement area prior to winding the tube, e.g., by means of a squeezing means that squeezes the product to be packed out of the displacement area. In this case, the displacement area may be clearly larger than the winding area. Instead, however, it is also possible to achieve the displacement of the material in the tube by the winding itself. In this case, the displacement area is substantially as large as the winding area or slightly larger. This can be implemented by the fact that the embracing, means is under the effect of a tensile force during winding. While the embracing means is wound around the tube filled with the material, a radially acting force is applied onto the surface of the tube because of the tensile force in the embracing means. Thereby, the material is laterally pressed out of the displacement area in a manner similar to squeezing. A combination of the two steps is also possible, e.g., by partially displacing the material in the tube by means of a squeezing tool and then performing the winding in that area, whereby the remaining material is simultaneously displaced. By this measure, it can be prevented that inadvertently, the tube filled with the material is unintentionally separated or damaged by the shearing forces produced during winding.

Preferably, the method according to the invention is supplemented by the method step of filling the tube with the material whereby the tube merchandise to be wound is actually produced. Further, the tube filled with the material can be transported further whereby it is possible to provide a continuous filling of the tube. The transport of the tube filled with the material can be achieved by filling, e.g., by the weight of the filled material drawing the filled tube in the direction of gravity. After the embracing means has been fixed in the displacement area, the tube is particularly separated in the displacement area.

Prior to separating, the tube can be embraced with the embracing means more than once so that the tightness of the tubular pack is not lost by separating. The tightness is preserved particularly by producing several loops during winding. To this end, the tube can be embraced particularly at least 5 times, preferably at least 20 times and, in a particularly preferred manner, at least 60 times when being wound with the embracing means.

By fixing the embracing means in the displacement area, at least one fixing area is produced. This fixing area may be identical to the displacement area. It is also possible, however, that fixing area and displacement area differ from each other. In principle, it would be sufficient to configure the fixing area approximately punctually so that particularly the ends of the embracing means are fixed to avoid that the embracing means is wound off the tube.

Preferably, the fixing is effected by a material engagement. The material engagement is particularly effected by a supply of energy. Similar to a soldering iron, the energy supply can be effected by heat and/or, for example, by friction by means of frictional elements. Additionally or alternatively, the energy supply can also be effected in a contactless manner. Similar to arc welding, this can be done by electric current. For a two-dimensional material engagement, an energy supply via ultrasound presents itself. Particularly if the embracing means consists at least partially, of synthetic fiber, it is melted on by the energy supply so that a material engagement with itself and/or the tube is effected. In a preferred embodiment, the energy supply is continued until the tube is separated in addition to the fixing of the embracing means. Thereby, it is possible that a melted-on embracing means also covers the point of separation. A tubular pack is created which, together with the embracing means, completely envelops the material in a material-engaging manner. Thus, the filled material is completely isolated with respect to the environment. The preferably precisely one winding step takes place between two tubular packs to be manufactured. Thus, the sealing of two ends of two different tubular packs is effected by a single winding step.

The separation is preferably effected in the particularly single winding area. When two fixing areas are arranged in the winding area, the separation is preferably effected between the two fixing areas so that a part of the wound embracing means may unwind. Thus, the wound embracing means can be additionally used as a holding element, particularly as a sausage suspending means. If only a single fixing area is arranged in the winding area, the separation is preferably effected in the fixing area so that two tight ends of two different tubular packs can be produced by particularly few operational steps. Particularly if this separation is effected by a heat supply in which the embracing means is partially melted on, the interface is sealed by the melted-on embracing means, so that the material disposed in the tube cannot come into contact with the environment.

Particularly, the tube consists of an elastomeric material so that material bulgings, foldings and the like of the tubular envelope can be substantially avoided. The tubular envelope particularly consists of a similar, preferably of the same material so that additionally, a bonding of the embracing means with the tubular envelope can be effected in a material-engaging manner.

In a preferred embodiment, the tubular pack is provided with a holding element. This can be done by arranging the holding element between the embracing means and the tube. The holding element, which is a hook, sausage suspending means or the like, can be connected particularly firmly with the tubular pack by an energy supply effecting a material engagement in particular.

Further, the invention relates to a device for manufacturing tubular packs filled with a material, particularly with a food, particularly for carrying out the afore-described method. The device according to the invention comprises a winding means by means of which an embracing means can be wound around a tube filled with the material. The winding is effected in at least two displacement areas and takes place there with an embracing angle of more than 360°. Additionally, the device comprises a fixing means by means of which the embracing means is fixed in the displacement area. At least one fixing area is produced so that the tubular pack is sealed tightly. Corresponding to the afore-described method according to the invention, the device according to the invention produces a particularly tight tubular pack in a particularly simple manner, filled tubes of a great variety of diameters being able to be used as base material. In particular, the device comprises a tension element by which a tensile force can be exerted upon the embracing means. The impressed tensile force is particularly so great that a displacement of the material in the tube can be effected upon winding the tube filled with the material with the embracing means. Thereby, it is effected that the winding means simultaneously takes over the function of a displacing means. Further, the device according to the invention may comprise a displacing means that displaces the material in the tube, particularly by means of a squeezing tool, so that substantially no material remains in the displacement area. The displacing means may also be used to only partially displace the material in the tube. Thereby, it can be prevented that the tube is unintentionally separated when the winding means is wound around the tube.

The embracing means used in the winding means consists at least partially of synthetic fiber that is particularly meltable. With such an embracing means, it is particularly possible to achieve a material engagement. This material engagement can be achieved in that the fixing means comprises an energy-supply means. By means of this energy supply means, heat and/or friction and/or ultrasound and/or electric current can be introduced into the displacement area whereby, in a preferred embodiment, the meltable embracing means melts at least partially and enters into a material engagement with itself and/or with the tube. When the energy supply means supplies the energy long enough or intensively enough, it is also possible to achieve a separation thereby. It is also possible that the device additionally comprises a separating means for separating the tube in the displacement area.

The device according to the invention is particularly employed to carry out the method according to the invention. Thereby, it is possible to achieve the advantages and effects of the method according to the invention by means of the device according to the invention, as has been described above.

Further, the invention relates to a tubular pack as it is particularly employed as a product of the manufacturing method according to the invention. The tubular pack according to the invention comprises a material, particularly a food, which is enveloped by a tube. The tubular envelope comprises a first end and a second end each of which is arranged in a displacement area. The displacement area in which there is substantially no material is produced by an embracing means wound around the displacement area. The first end and the second end of the tube respectively end in the displacement area in a tapering manner. This means that the inside of the tube-has no contact with the environment.

This means that there is no tube portion flaring again, away from the enveloped material, after a point of maximum tapering. By this configuration of the tubular pack according to the invention, the entire length of the tube, is employed for enveloping the material. By the optimum use of the entire tube length as an envelope for the filled material, the required tube length necessary for enveloping the material can be reduced whereby the manufacturing costs are reduced. Further, a particular tightness of the tubular pack is achieved by the wound embracing means. Particularly if the embracing means is fixed in the displacement area, it is ensured that the tubular pack remains tight, too.

In a preferred embodiment, the embracing means is fixed in a material-engaging manner. The material engagement is particularly effected such that the first and/or the second end of the tube are enclosed in a cap-like manner. In a particularly preferred embodiment, the first end and/or the second end end in a material-engaging manner, i.e., the inside of the tube is connected with itself at the end thereof. Thereby, the tightness of the tubular pack according to the invention is clearly increased.

### BRIEF DESCRIPTION OF THE DRAWINGS

Hereinafter, the invention is explained in detail with reference to the accompanying drawings in which:

FIG. 1 shows a schematic side view of a device according to the invention,

FIG. 2 shows a schematic top view of an example for a winding means, and

FIG. 3 a schematic sectional view of a tubular pack according to the invention.

### DETAILED DESCRIPTION OF PREFERRED EMBODIMENTS OF THE INVENTION

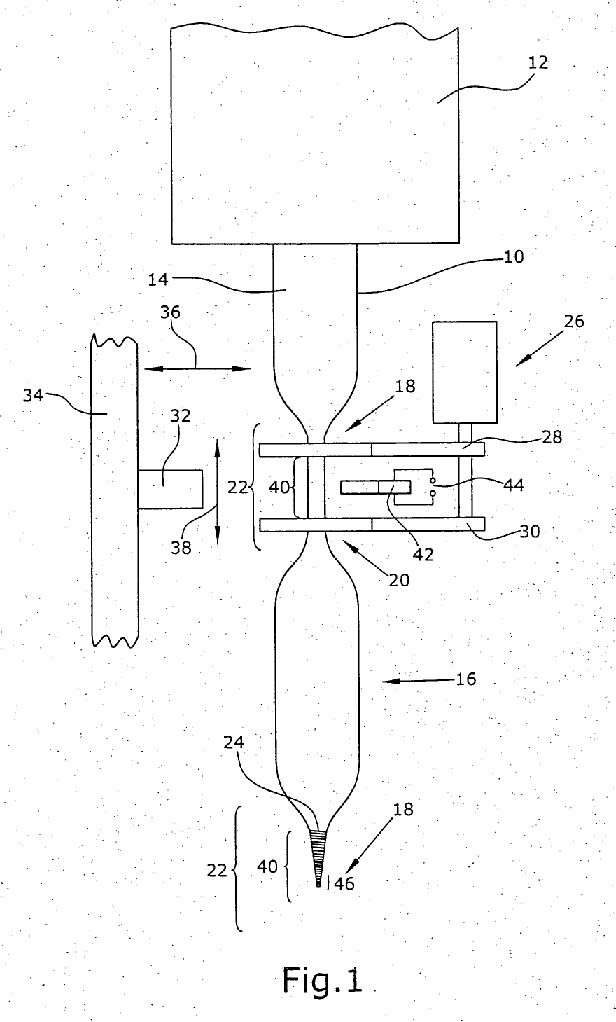

FIG. 1 shows that a tube 10 is filled with a material, particularly a food, within a filling means 12. By the dead weight of a filled tube 14, it is transported downward without requiring a transport device of its own. The filled tube 14 is separated into individual tubular packs 16 comprising a first end 18 and a second end 20 by which the tubular pack 16 is limited.

The tube 14 filled with the material comprises a displacement area 22 that is to be wound with an embracing means 24. To this end, the filled tubular pack 14, in the illustrated embodiment, is first gripped, in the displacement area 22 by means of a displacing means 26 comprising two grippers 28,30 formed as two clamps. Subsequently, the grippers 28,30 are moved away from each other so that a large part of the material is displaced out of the displacement area 22.

Subsequently, a winding means 32 mounted at a carrier 34 is conducted in the direction of the arrow 36 into the displacement area 22 located between the grippers 28,30. The winding means 32 is height-adjustable in the direction of the arrow 38 so that particularly the entire displacement area 22 can be largely wound. A suitable winding means 32 is described, for example, in GB 1 306 086. By the winding, a winding area 40 is created which is at least a part of the displacement area 22. By the winding, an additional displacement takes place in the illustrated embodiment by the embracing means 24 constricting the displacement area 22 so that material still left is additionally displaced out of the displacement area 22.

The embracing means 24 is fixed by means of a fixing means 42. In the illustrated embodiment, the fixing means 42 has a clamp-like configuration and can encompass the tube or the tubular envelope 10 in the displacement area 22, particularly in the winding area 40. The fixing means 42 is connected to an electric network 44 and is able to radially introduce a heat into the displacement area 22 in a manner similar to that of a circularly configured soldering iron. Thereby, the embracing means 24, which particularly is a meltable synthetic fiber, is melted on so that a fixing area 46 is created in which the embracing means 24 is connected with itself in a material-engaging manner. Thereby, the tubular pack 16 is tightly sealed at its ends 18,20.

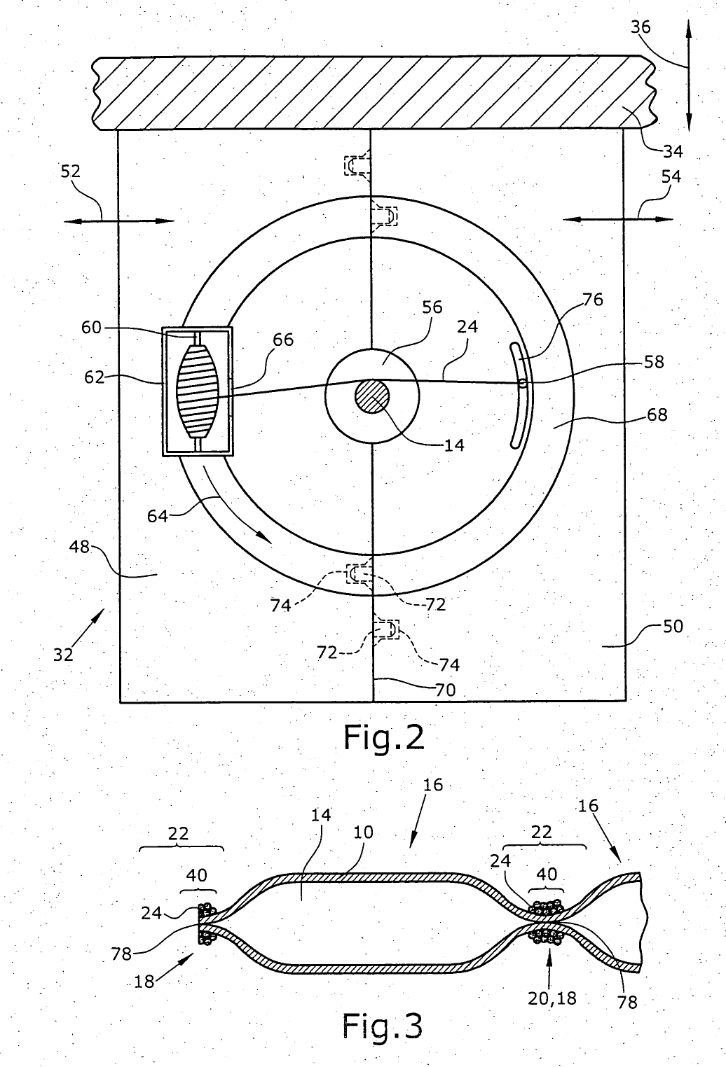

FIG. 2 shows how the tube 14 possibly filled with material can be wound with an embracing means 24. To this end, the winding means 32 illustrated herein comprises two plates 48,50 which are laterally displaceable in the direction of the arrows, 52,54. By the lateral displaceability of the plates 48,50, it is possible that the winding means 32 can encompass the displacement area 22 of the filled tube 14. After encompassing, the tube 14 possibly still filled with material is arranged inside an opening 56.

The winding means. 32 comprises a tension element 58 holding fast the one end of the embracing means 24. The rest of the embracing means 24 is wound on a shaft 60 rotatably borne in a shuttle 62. The shuttle 62 rotates around the filled tube 14 in the direction of the arrow 64 in order to wind it thereby. The embracing means 24 is guided through a recess 66 in the shuttle 62. Instead of a rotation, the shuttle 62 may also be shot from one side of the winding means 32 to the other side as is otherwise common in textile technology in order to wind the filled tube 14. In doing so, however, the shuttle 62 should be slightly offset relative to the filled tube 14 with every shot so that the shuttle 62 cannot collide with the filled tube 14.

The shuttle 62 may comprise, e.g., a drive unit by means of which it is able to move around the filled tube 14 on a kind of rail track so that the shuttle 62 is able to rotate about the filled tube 14. The shuttle 62, however, may also be connected with a ring 68 which is set in rotation by a drive means. The plates 48,50 as well as the divided ring 68 comprise centering bolts 72 and centering bores 74, respectively, so that the movement over a seam 70 between the plates 48,50 is effected as unproblematically as possible.

For a better regulation of the tensile force, applied onto the embracing means 24 by the tension element 58, particularly for constricting the filled tube 14, the tension element 58 is adjustably arranged in a groove 76. Depending on the position of the tension element 58 in the groove 76, the tensile force exerted onto the embracing means 24 is stronger or weaker.

By an appropriate coordination of the height adjustment of the winding means and the movement of the shuttle 62, different winding types can be realized. It is possible, for example, to produce several loops which result in parallel and/or crossed courses of the embracing means 24 when they are arranged next to and/or above each other. Particularly if more than one embracing means 24 is used at a time, particularly advantageous knot and weaving formations can be produced.

FIG. 3 shows the tubular pack 16 as it can be particularly produced by the method and the device according to the invention, respectively. The tube 10 of the tubular pack 16 is filled with a material, which is a food in particular. The first end 18 and the second end 20 of the tubular pack 16 are located in a displacement area 22 wound with an embracing means 24. The tubular pack 16 is limited by its ends 18,20 defined by an area or point 78 of maximum tapering of the tubular envelope 10. Thereby, the ends 18,20 of the tubular pack 16 respectively end in the displacement areas 22 in a tapering manner. When the individual tubular packs 16 are separated from each other, the separation is particularly effected at the very point 78 of maximum tapering. This means that the second end 20 of the tubular pack 16 abuts on the first end 18 of another tubular pack 16 before a separation has been effected. The tubular pack 16 is particularly tightly sealed in the displacement areas 22 by fixing the embracing means 24, the fixing being effected particularly in a material-engaging manner. This results in that the ends 18,20 of the tubular pack 16 end in a material-engaging manner in particular.

Although the invention has been described and illustrated with reference to specific illustrative embodiments thereof, it is not, intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

1. Method for manufacturing tubular packs filled with a material, particularly a food, with the following steps:
winding a tube filled with the material with an embracing means and an embracing angle of more than 360° in at least two displacement areas in order to form the tubular pack, precisely one winding area being produced in the respective displacement area, and
fixing the embracing means in the displacement area for producing at least one fixing area so that the tubular pack is sealed tightly.

2. Method according to claim 1, wherein the winding effects a displacement of the material in the tube so that substantially no material remains in the displacement areas.

3. Method according to claim 1, wherein the material in the tube is displaced prior to winding so that substantially no material remains in the displacement areas.

4. Method according to claim 1, wherein prior to winding, the method steps of
filling the tube with the material and/or
transporting the tube filled with the material and/or,
after fixing, the method step of
separating the tube in the displacement area are performed.

5. Method according to claim 4, wherein two fixing areas are produced in the displacement area between which a separation is effected.

6. Method according to claim 1, wherein the fixing is effected in a material-engaging manner, particularly by a supply of energy in the form of heat and/or friction and/or ultrasound and/or electric current.

7. Method according to claim 6, wherein the supply of energy is effected until not only the fixing, but also the separation has been performed.

8. Method according to claim 6, wherein a material engagement of the embracing means with itself and/or with the tube is produced by fixing.

9. Device for manufacturing tubular packs filled with a material, particularly a food, comprising
a winding means for winding a tube filled with the material in at least two displacement areas with an embracing means and an embracing angle of more than 360°, and
a fixing means for fixing the embracing means in the displacement area for producing at least one fixing area so that the tubular pack is tightly sealed.

10. Device according to claim 9, characterized in that the winding means comprises a tension element exerting a tensile force upon the embracing means, which is particularly so large that, upon winding the tube filled with the material with the embracing means, a displacement of the material in the tube is effected so that substantially no material is left in the displacement areas.

11. Device according to claim 9, characterized by a displacing means which, by means of a squeezing tool in particular, produces a displacement of the material in the tube so that substantially no material is left in the displacement area.

12. Device according to claim 9, characterized by a providing means for arranging a holding element, particularly a sausage suspending means, at least partially on the tube top side in a portion of the displacement area.

13. Device according to claim 9, characterized in that the fixing means comprises an energy supply means for introducing energy into the displacement area, particularly in the form of heat and/or friction and/or ultrasound and/or electric current.

14. Device according to claim 9, characterized by a separating means for separating the tube in the displacement area.

15. Tubular pack, particularly producible by a method according to claim 1, comprising
a tube enveloping a material, particularly a food,
a first end of the tube arranged in a first displacement area wound with an embracing means, and
a second end of the tube arranged in a second displacement area wound with the embracing means,
the first end and the second end each ending in the displacement areas in a tapering manner.

16. Tubular pack according to claim 15, characterized in that the embracing means is fixed in the displacement area so that the tubular pack is tightly sealed.

17. Tubular pack according to claim 15, characterized in that the first end and/or the second ends end in a material-engaging manner.
